(19) United States
(12) Patent Application Publication
(10) Pub. No.: US 2025/0040947 A1
(43) Pub. Date: Feb. 6, 2025

(54) INTRAVASCULAR LITHOTRIPSY DEVICE WITH PLASMA GENERATOR INCLUDING POLYMERIC MATERIAL

(71) Applicant: BOLT MEDICAL, INC., Carlsbad, CA (US)

(72) Inventor: Eric Schultheis, San Clemente, CA (US)

(21) Appl. No.: 18/741,023

(22) Filed: Jun. 12, 2024

Related U.S. Application Data
(60) Provisional application No. 63/516,938, filed on Aug. 1, 2023.

(51) Int. Cl. A61B 17/22; A61B 17/00

(52) U.S. Cl. CPC A61B 17/22012; A61B 17/00234; A61B 2017/00154; A61B 2017/00305; A61B 2017/00477; A61B 2017/00743; A61B 2017/00778; A61B 2017/00836; A61B 2017/00889; A61B 2017/00907; A61B 2017/00938; A61B 2017/00942; A61B 2017/00946; A61B 2017/22008; A61B 2017/22025

(57) ABSTRACT

A catheter system (100) for treating a treatment site (106) within or adjacent to a vessel wall (108A) of a blood vessel (108) includes an energy source (124), an energy guide (122A), and a guidewire lumen (118). The energy source (124) generates energy. The energy guide (122A) is configured to selectively receive the energy from the energy source (124). The energy guide (122A) includes a guide distal end (122D). The energy that is received by the energy guide (122A) is emitted from the guide distal end (122D). The guidewire lumen (118) having an outer surface (218S). At least the guide distal end (122D) of the energy guide (122A) is positioned adjacent to the outer surface (218S) of the guidewire lumen (118) A portion of the guidewire lumen (118) encompasses a plasma generator (133) that is positioned near the guide distal end (122D) of the energy guide (122A). The plasma generator (133) is positionable near the treatment site (106). The plasma generator (133) is formed from a polymeric material.

### RELATED APPLICATION

This application is related to and claims priority on U.S. Provisional Patent Application Ser. No. 63/516,938, filed on Aug. 1, 2023, and entitled “INTRAVASCULAR LITHOTRIPSY DEVICE WITH EMITTERS INCLUDING POLYMERIC MATERIAL”. To the extent permissible, the contents of U.S. Provisional Patent Application Ser. No. 63/516,938, are incorporated in their entirety herein by reference.

### BACKGROUND

Vascular lesions within vessels in the body can be associated with an increased risk for major adverse events, such as myocardial infarction, embolism, deep vein thrombosis, stroke, and the like. Severe vascular lesions can be difficult to treat and achieve patency for a physician in a clinical setting.

Vascular lesions may be treated using interventions such as drug therapy, balloon angioplasty, atherectomy, stent placement, vascular graft bypass, to name a few. Such interventions may not always be ideal or may require subsequent treatment to address the lesion.

### SUMMARY

The present invention is directed toward a catheter system for placement within a blood vessel having a vessel wall. The catheter system can be used by an operator for treating a treatment site within or adjacent to the vessel wall. In various embodiments, the catheter system includes an energy source, an energy guide, and a guidewire lumen. The energy source generates energy. The energy guide is configured to selectively receive the energy from the energy source, the energy guide including a guide distal end, the energy that is received by the energy guide being emitted from the guide distal end. The guidewire lumen has an outer surface. At least the guide distal end of the energy guide is positioned adjacent to the outer surface of the guidewire lumen. A portion of the guidewire lumen encompasses a plasma generator that is positioned near the guide distal end of the energy guide. The plasma generator is positionable near the treatment site. The plasma generator is formed from a polymeric material.

In some embodiments, the outer surface of the guidewire lumen includes a groove. In certain embodiments, at least the guide distal end of the energy guide is positioned within the groove formed along the outer surface of the guidewire lumen.

In certain embodiments, the plasma generator is at least partially formed from one of plastic, polyimide, nylon, and polyether block amide.

In many embodiments, the plasma generator is positioned spaced apart from the guide distal end of the energy guide.

In various embodiments, the energy that is received by the energy guide is emitted from the guide distal end and contacts the plasma generator so that plasma is generated adjacent to the plasma generator.

In some embodiments, the plasma generation creates an acoustic wave that is directed away from the plasma generator. In certain embodiments, the acoustic wave imparts pressure adjacent to the vessel wall at the treatment site.

In some embodiments, a polymeric filler is added to the polymeric material of the plasma generator.

In certain embodiments, the polymeric filler includes one or more of titanium dioxide, bismuth, barium sulfate, gold nanoparticles, silver nanoparticles, and tungsten particles.

In many embodiments, the catheter system further includes a catheter shaft and a balloon that is coupled to the catheter shaft. The balloon includes a balloon wall that defines a balloon interior. The balloon is configured to retain a catheter fluid within the balloon interior. The guide distal end of the energy guide and the plasma generator are positioned within the balloon interior.

In some embodiments, the energy that is received by the energy guide is emitted from the guide distal end and contacts the plasma generator so that plasma is generated in the catheter fluid retained within the balloon interior.

In certain embodiments, the guide distal end of the energy guide is angled relative to the outer surface of the guidewire lumen so that the energy emitted from the guide distal end is emitted toward the outer surface of the guidewire lumen within the groove. In some embodiments, a portion of the outer surface of the guidewire lumen encompasses the plasma generator.

In certain embodiments, the guide distal end of the energy guide is angled relative to the outer surface of the guidewire lumen at between approximately 5 degrees and 45 degrees relative to a flat, perpendicular configuration.

In some embodiments, the plasma generator extends outwardly away from the outer surface of the guidewire lumen and includes an angled face so that the energy emitted from the guide distal end of the energy guide is emitted toward the angled face of the plasma generator and redirected toward the treatment site.

In certain embodiments, the angled face of the plasma generator is angled relative to the outer surface of the guidewire lumen at between approximately 5 degrees and 45 degrees relative to a flat, perpendicular configuration.

In many embodiments, the catheter system further includes a system controller including a processor that controls the energy source so that the energy from the energy source is selectively directed to the energy guide.

In various embodiments, the energy source is a light source that generates pulses of light energy.

In some embodiments, the light source is a laser.

In many embodiments, the energy guide includes an optical fiber.

The present invention is also directed toward a catheter system for treating a treatment site within or adjacent to a vessel wall of a blood vessel, including an energy source that generates energy; a catheter shaft and a balloon that is coupled to the catheter shaft, the balloon including a balloon wall that defines a balloon interior, the balloon being configured to retain a catheter fluid within the balloon interior; an energy guide that is configured to selectively receive the energy from the energy source, the energy guide including a guide distal end that is positioned within the balloon interior, the energy that is received by the energy guide being emitted from the guide distal end; and a guidewire lumen having an outer surface, at least the guide distal end of the energy guide being positioned adjacent to the outer surface of the guidewire lumen, a portion of the guidewire lumen encompassing a plasma generator that is positioned within the balloon interior and near the guide distal end of the energy guide, the plasma generator being positionable near the treatment site, the plasma generator being formed at least partially from a polymeric material that includes at least one of plastic, polyimide, nylon, and polyether block amide.

The present invention is further directed toward a method for treating a treatment site within or adjacent to a vessel wall of a blood vessel, including the steps of generating energy with an energy source; selectively receiving the energy from the energy source with an energy guide, the energy guide including a guide distal end; emitting the energy that is received by the energy guide from the guide distal end; positioning at least the guide distal end of the energy guide adjacent to an outer surface of a guidewire lumen, a portion of the guidewire lumen encompassing a plasma generator that is positioned near the guide distal end of the energy guide; and positioning the plasma generator near the treatment site, the plasma generator being formed from a polymeric material.

This summary is an overview of some of the teachings of the present application and is not intended to be an exclusive or exhaustive treatment of the present subject matter. Further details are found in the detailed description and appended claims. Other aspects will be apparent to persons skilled in the art upon reading and understanding the following detailed description and viewing the drawings that form a part thereof, each of which is not to be taken in a limiting sense. The scope herein is defined by the appended claims and their legal equivalents.

### BRIEF DESCRIPTION OF THE DRAWINGS

The novel features of this invention, as well as the invention itself, both as to its structure and its operation, will be best understood from the accompanying drawings, taken in conjunction with the accompanying description, in which similar reference characters refer to similar parts, and in which:

FIG. 1 is a simplified schematic cross-sectional view illustration of an embodiment of a catheter system in accordance with various embodiments, the catheter system including a guidewire lumen, and one or more energy guides that are positioned adjacent to the guidewire lumen, a portion of the guidewire lumen encompassing a plasma generator;

FIG. 2 is a simplified schematic end view illustration of a portion of an embodiment of the catheter system including an embodiment of the guidewire lumen and an energy guide that is positioned adjacent to the guidewire lumen;

FIG. 3A is a simplified schematic side view illustration of a portion of the guidewire lumen and a portion of the energy guide illustrated in FIG. 2, and an embodiment of the plasma generator which can be encompassed within a portion of the guidewire lumen;

FIG. 3B is a simplified schematic side view illustration of the portion of the guidewire lumen, the plasma generator, and the portion of the energy guide illustrated in FIG. 3A, which further illustrates plasma plume initiation;

FIG. 3C is a simplified schematic side view illustration of the portion of the guidewire lumen, the plasma generator, and the portion of the energy guide illustrated in FIG. 3A, which further illustrates plasma plume generation and resulting high-frequency acoustic waves;

FIG. 3D is a simplified schematic side view illustration of the portion of the guidewire lumen, the plasma generator, and the portion of the energy guide illustrated in FIG. 3A, which further illustrates cavitation bubble generation;

FIG. 4A is a simplified schematic side view illustration of a portion of another embodiment of the guidewire lumen, a portion of another embodiment of the energy guide, and another embodiment of the plasma generator which can be encompassed within the guidewire lumen;

FIG. 4B is a simplified schematic side view illustration of the portion of the guidewire lumen, the plasma generator, and the portion of the energy guide illustrated in FIG. 4A, which further illustrates plasma plume initiation; and

FIG. 4C is a simplified schematic top view illustration of the portion of the guidewire lumen, the plasma generator, and the portion of the energy guide illustrated in FIG. 4A, which further illustrates plasma plume generation and resulting high-frequency acoustic waves.

While embodiments of the present invention are susceptible to various modifications and alternative forms, specifics thereof have been shown by way of example and drawings, and are described in detail herein. It is understood, however, that the scope herein is not limited to the particular embodiments described. On the contrary, the intention is to cover modifications, equivalents, and alternatives falling within the spirit and scope herein.

### DESCRIPTION

Treatment of vascular lesions at treatment sites within a body of a patient can reduce major adverse events or death in affected subjects. As referred to herein, a major adverse event is one that can occur anywhere within the body due to the presence of a vascular lesion. Major adverse events can include, but are not limited to, major adverse cardiac events, major adverse events in the peripheral or central vasculature, major adverse events in the brain, major adverse events in the musculature, or major adverse events in any of the internal organs.

In various embodiments, the catheter systems and related methods disclosed herein can include a catheter configured to advance to a vascular lesion, such as a calcified vascular lesion or a fibrous vascular lesion, at a treatment site located within a body of a patient. In certain implementations, the “treatment site” can be located at or near a vessel wall of a blood vessel of the patient. Additionally, or in the alternative, in other implementations, the “treatment site” can be at or near a heart valve of the patient. Further, or in the alternative, in still other implementations, the “treatment site” can be at another suitable location within the body of the patient.

As used herein, the terms “treatment site”, “intravascular lesion” and “vascular lesion” are used interchangeably unless otherwise noted. The intravascular lesions and/or the vascular lesions are sometimes referred to herein as “lesions”.

Those of ordinary skill in the art will realize that the following detailed description of the present invention is illustrative only and is not intended to be in any way limiting. Other embodiments of the present invention will readily suggest themselves to such skilled persons having the benefit of this disclosure. Reference will now be made in detail to implementations of the present invention as illustrated in the accompanying drawings.

In the interest of clarity, not all of the routine features of the implementations described herein are shown and described. It will, of course, be appreciated that in the development of any such actual implementation, numerous implementation-specific decisions must be made in order to achieve the developer's specific goals, such as compliance with application-related and business-related constraints, and that these specific goals will vary from one implementation to another and from one developer to another. Moreover, it is appreciated that such a development effort might be complex and time-consuming, but would nevertheless be a routine undertaking of engineering for those of ordinary skill in the art having the benefit of this disclosure.

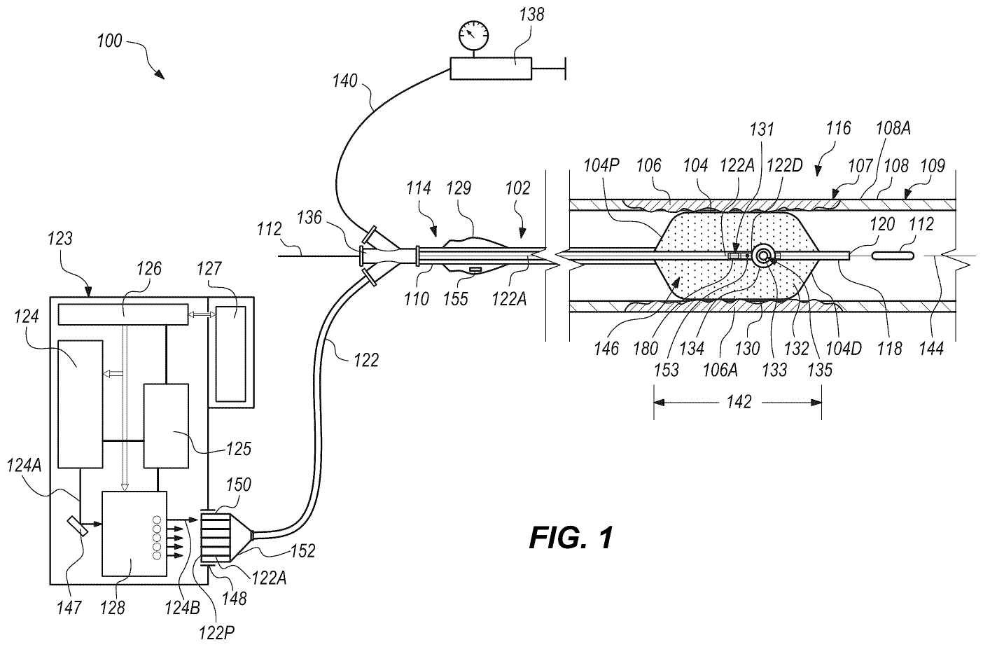

The catheter systems disclosed herein can include many different forms. Referring now to FIG. 1, a simplified schematic cross-sectional view illustration is shown of a catheter system 100 in accordance with various embodiments. The catheter system 100 is suitable for imparting high-frequency mechanical acoustic waves and/or pressure waves to induce fractures in one or more vascular lesions at treatment sites within or adjacent to a vessel wall of a blood vessel or on or adjacent to a heart valve within a body of a patient. In the embodiment illustrated in FIG. 1, the catheter system 100 can include (i) a catheter 102 including one or more of an inflatable balloon 104 (sometimes referred to herein as a “balloon”), a catheter shaft 110, a guidewire 112, an energy guide bundle 122 including one or more energy guides 122A, a source manifold 136, a fluid pump 138, a handle assembly 129, and an energy emitting system 131 (also referred to herein as an “emitter system”) including one or more emitter stations 180 and/or one or more emitters 135; and (ii) a system console 123 including one or more of an energy source 124 (such as a light source and/or a laser source in certain non-exclusive embodiments), a power source 125, a system controller 126, a graphic user interface 127 (a “GUI”), and a multiplexer 128. Alternatively, the catheter system 100, the catheter 102 and/or the system console 123 can include more components or fewer components than those specifically illustrated and described in relation to FIG. 1. For example, in certain non-exclusive alternative embodiments, the catheter system 100 and/or the system console 123 can be designed without the GUI 127 and/or the multiplexer 128.

The catheter 102 is configured to move to a treatment site 106 at any suitable location within a body 107 of a patient 109. In some implementations, the treatment site 106 can be within or adjacent to a vessel wall 108A of a blood vessel 108 within the body 107 of the patient 109. Alternatively, in other implementations, the catheter 102 can be used at a treatment site 106 within or adjacent to a heart valve within the body 107 of the patient 109. The treatment site 106 can include one or more vascular lesions 106A such as calcified vascular lesions, for example. Additionally, or in the alternative, the treatment site 106 can include vascular lesions 106A such as fibrous vascular lesions.

In certain embodiments, the balloon 104 can be coupled to the catheter shaft 110. The balloon 104 can include a balloon proximal end 104P and a balloon distal end 104D. The catheter shaft 110 can extend from a proximal portion 114 of the catheter system 100 to a distal portion 116 of the catheter system 100. The catheter shaft 110 can include a longitudinal axis 144. The catheter 102 and/or the catheter shaft 110 can also include a guidewire lumen 118 which is configured to move over the guidewire 112. As utilized herein, the guidewire lumen 118 defines a conduit through which the guidewire 112 extends. The catheter shaft 110 can further include an inflation lumen (not shown) and/or various other lumens for various other purposes. In some embodiments, the catheter 102 can have a distal end opening 120 and can accommodate and be tracked over the guidewire 112 as the catheter 102 is moved and positioned at or near the treatment site 106. In some embodiments, the balloon proximal end 104P can be coupled to the catheter shaft 110, and the balloon distal end 104D can be coupled to the guidewire lumen 118.

The balloon 104 includes a balloon wall 130 that defines a balloon interior 146. The balloon 104 can be selectively inflated with a catheter fluid 132 to expand from a deflated state suitable for advancing the catheter 102 through a patient's vasculature, to an inflated state (as shown in FIG. 1) suitable for anchoring the catheter 102 in position relative to the treatment site 106. Stated in another manner, when the balloon 104 is in the inflated state, the balloon wall 130 of the balloon 104 is configured to be positioned substantially adjacent to the treatment site 106.

As referred to herein, each emitter station 180 can include one or more emitters 135 that are positioned at approximately the same longitudinal position within the balloon 104. Each emitter 135 includes at least a guide distal end 122D of one of the energy guides 122A and a corresponding plasma generating structure 133 (also referred to herein as a “plasma generator”) that cooperate to generate a plasma within the balloon 104. Since the emitter system 131 can include one or more emitter stations 180, it is appreciated that the catheter system 100 and/or the emitter system 131 can include any suitable number of emitters 135, from only one emitter 135, to greater than 30 emitters 135.

As an overview, in various embodiments, each of the emitters 135 of the catheter system 100 can be formed, at least in part, from one or more polymeric materials. More specifically, the present invention is directed toward a polymeric emitter 135 for use within an intravascular lithotripsy device that generates an acoustic wave by a laser light interaction. The invention includes an energy guide 122A, such as an optical fiber in certain non-exclusive embodiments, that is attached to and/or positioned adjacent to the guidewire lumen 118. In many embodiments, the plasma generator 133, which can be encompassed within a portion of the guidewire lumen 118 that is spaced apart from the guide distal end 122D of the energy guide 122A, can be formed from one or more polymeric materials.

In some embodiments, the guidewire lumen 118 and/or the plasma generator 133 is at least partially formed from polymeric materials such as polyether block amide (such as PEBAX™), polyimide, plastic, nylon, or other thermoplastic, and can further be configured to have a thin wall to minimize crossing profile. Polymeric fillers such as titanium dioxide, bismuth, barium sulfate, gold nanoparticles, silver nanoparticles, tungsten particles or any other metallic materials may also be added to the polymeric or thermoplastic material of the guidewire lumen 118 and/or the plasma generator 133 to increase laser absorption and therefore optimize conversion efficiency between the laser energy and acoustic output. In certain embodiments, energy, such as laser energy, that is directed out from the guide distal end 122D of the energy guide 122A comes into contact with the polymeric materials on the guidewire lumen 118 (a portion of which encompasses the plasma generator 133 in various embodiments) at a distance away from the guide distal end 122D of the energy guide 122A. The light interaction with the polymeric material initiates a plasma plume substantially adjacent to the plasma generator 133. The plasma plume grows in size in proportion with the energy that is delivered through the emitter 135 and/or the energy guide 122A. In various implementations, the creation of the plasma plume creates a high-frequency mechanical acoustic wave that is directed away from the plasma generator 133 and toward the vascular lesions 106A at the treatment site 106. Thus, the plasma generation generates an acoustic wave and/or a pressure wave that imparts pressure adjacent to the vascular lesions 106A at the treatment site 106. A cavitation bubble can also be created by the acoustic wave as it propagates away from the plasma generator 133 and toward the vascular lesions 106A at the treatment site 106.

The balloon 104 suitable for use in the catheter system 100 includes those that can be passed through the vasculature of a patient 109 when in the deflated state. In some embodiments, the balloons 104 are made from silicone. In other embodiments, the balloon 104 can be made from materials such as polydimethylsiloxane (PDMS), polyurethane, polymers such as a polyether block amide (such as PEBAX™) material, nylon, or any other suitable material.

The balloon 104 can have any suitable diameter (in the inflated state). In various embodiments, the balloon 104 can have a diameter (in the inflated state) ranging from less than one millimeter (mm) up to 25 mm. In some embodiments, the balloon 104 can have a diameter (in the inflated state) ranging from at least 1.5 mm up to 14 mm. In certain embodiments, the balloon 104 can have a diameter (in the inflated state) ranging from at least two mm up to five mm.

In some embodiments, the balloon 104 can have a length 142 ranging from at least three mm to 300 mm. More particularly, in certain embodiments, the balloon 104 can have a length 142 ranging from at least eight mm to 200 mm. It is appreciated that a balloon 104 having a relatively longer length can be positioned adjacent to larger treatment sites 106, and, thus, may be usable for imparting acoustic waves and/or pressure waves onto and inducing fractures in larger vascular lesions 106A or multiple vascular lesions 106A at precise locations within the treatment site 106. It is further appreciated that a longer balloon 104 can also be positioned adjacent to multiple treatment sites 106 at any one given time.

The balloon 104 can be inflated to inflation pressures of between approximately one atmosphere (atm) and 70 atm. In some embodiments, the balloon 104 can be inflated to inflation pressures of from at least 20 atm to 60 atm. In other embodiments, the balloon 104 can be inflated to inflation pressures of from at least six atm to 20 atm. In still other embodiments, the balloon 104 can be inflated to inflation pressures of from at least three atm to 20 atm. In yet other embodiments, the balloon 104 can be inflated to inflation pressures of from at least two atm to ten atm.

The balloon 104 can have various shapes, including, but not to be limited to, a conical shape, a square shape, a rectangular shape, a spherical shape, a conical/square shape, a conical/spherical shape, an extended spherical shape, an oval shape, a tapered shape, a bone shape, a stepped diameter shape, an offset shape, or a conical offset shape. In some embodiments, the balloon 104 can include a drug eluting coating or a drug eluting stent structure. The drug eluting coating or drug eluting stent structure can include one or more therapeutic agents including anti-inflammatory agents, anti-neoplastic agents, anti-angiogenic agents, and the like.

The catheter fluid 132 can be a liquid or a gas. Some examples of the catheter fluid 132 suitable for use can include, but are not limited to one or more of water, saline, contrast medium, fluorocarbons, perfluorocarbons, gases, such as carbon dioxide, or any other suitable catheter fluid 132. In some embodiments, the catheter fluid 132 can be used as a base inflation fluid. In some embodiments, the catheter fluid 132 can include a mixture of saline to contrast medium in a volume ratio of approximately 50:50. In other embodiments, the catheter fluid 132 can include a mixture of saline to contrast medium in a volume ratio of approximately 25:75. In still other embodiments, the catheter fluid 132 can include a mixture of saline to contrast medium in a volume ratio of approximately 75:25. However, it is understood that any suitable ratio of saline to contrast medium can be used. The catheter fluid 132 can be tailored on the basis of composition, viscosity, and the like so that the rate of travel of the acoustic waves and/or the pressure waves are appropriately manipulated. In certain embodiments, the catheter fluids 132 suitable for use are biocompatible. A volume of catheter fluid 132 can be tailored by the chosen energy source 124 and the type of catheter fluid 132 used.

In some embodiments, the contrast agents used in the contrast media can include, but are not to be limited to, iodine-based contrast agents, such as ionic or non-ionic iodine-based contrast agents. Some non-limiting examples of ionic iodine-based contrast agents include diatrizoate, metrizoate, iothalamate, and ioxaglate. Some non-limiting examples of non-ionic iodine-based contrast agents include iopamidol, iohexol, ioxilan, iopromide, iodixanol, and ioversol. In other embodiments, non-iodine-based contrast agents can be used. Suitable non-iodine containing contrast agents can include gadolinium (III)-based contrast agents. Suitable fluorocarbon and perfluorocarbon agents can include, but are not to be limited to, agents such as the perfluorocarbon dodecafluoropentane (DDFP, C₅F₁₂).

The catheter fluids 132 can include those that include absorptive agents that can selectively absorb light in the ultraviolet region (e.g., at least ten nanometers (nm) to 400 nm), the visible region (e.g., at least 400 nm to 780 nm), or the near-infrared region (e.g., at least 780 nm to 2.5 micrometers (μm)) of the electromagnetic spectrum. Suitable absorptive agents can include those with absorption maxima along the spectrum from at least ten nm to 2.5 μm. Alternatively, the catheter fluids 132 can include those that include absorptive agents that can selectively absorb light in the mid-infrared region (e.g., at least 2.5 μm to 15 μm), or the far-infrared region (e.g., at least 15 μm to one mm) of the electromagnetic spectrum. In various embodiments, the absorptive agent can be those that have an absorption maximum matched with the emission maximum of the laser used in the catheter system 100. By way of non-limiting examples, various lasers usable in the catheter system 100 can include neodymium:yttrium-aluminum-garnet (Nd:YAG-emission maximum=1064 nm) lasers, holmium:YAG (Ho:YAG-emission maximum=2.1 μm) lasers, or erbium:YAG (Er:YAG-emission maximum=2.94 μm) lasers. In some embodiments, the absorptive agents can be water-soluble. In other embodiments, the absorptive agents are not water-soluble. In some embodiments, the absorptive agents used in the catheter fluids 132 can be tailored to match the peak emission of the energy source 124. Various energy sources 124 having emission wavelengths of at least ten nanometers to one millimeter are discussed elsewhere herein.

The catheter shaft 110 of the catheter 102 can be coupled to the plurality of energy guides 122A of the energy guide bundle 122 that are in optical communication with the energy source 124. The energy guide(s) 122A can be disposed along the catheter shaft 110 and within the balloon 104. Each of the energy guides 122A can have a guide distal end 122D that is at any suitable longitudinal position relative to the length 142 of the balloon 104 and/or relative to a length of the guidewire lumen 118.

In some embodiments, each energy guide 122A can be an optical fiber and the energy source 124 can be a laser. The energy source 124 can be in optical communication with the energy guides 122A at the proximal portion 114 of the catheter system 100. More particularly, the energy source 124 can selectively and/or alternatively be in optical communication with each of the energy guides 122A due to the presence and operation of the multiplexer 128.

In some embodiments, the catheter shaft 110 can be coupled to multiple energy guides 122A such as a first energy guide, a second energy guide, a third energy guide, etc., which can be disposed at any suitable positions about and/or relative to the guidewire lumen 118 and/or the catheter shaft 110. For example, in certain non-exclusive embodiments, two energy guides 122A can be spaced apart from one another by approximately 180 degrees about the circumference of the guidewire lumen 118 and/or the catheter shaft 110; three energy guides 122A can be spaced apart from one another by approximately 120 degrees about the circumference of the guidewire lumen 118 and/or the catheter shaft 110; four energy guides 122A can be spaced apart from one another by approximately 90 degrees about the circumference of the guidewire lumen 118 and/or the catheter shaft 110; five energy guides 122A can be spaced apart from one another by approximately 72 degrees about the circumference of the guidewire lumen 118 and/or the catheter shaft 110; six energy guides 122A can be spaced apart from one another by approximately 60 degrees about the circumference of the guidewire lumen 118 and/or the catheter shaft 110; eight energy guides 122A can be spaced apart from one another by approximately 45 degrees about the circumference of the guidewire lumen 118 and/or the catheter shaft 110; or ten energy guides 122A can be spaced apart from one another by approximately 36 degrees about the circumference of the guidewire lumen 118 and/or the catheter shaft 110. Still alternatively, multiple energy guides 122A need not be uniformly spaced apart from one another about the circumference of the guidewire lumen 118 and/or the catheter shaft 110. More particularly, it is further appreciated that the energy guides 122A can be disposed uniformly or non-uniformly about the guidewire lumen 118 and/or the catheter shaft 110 to achieve the desired effect in the desired locations.

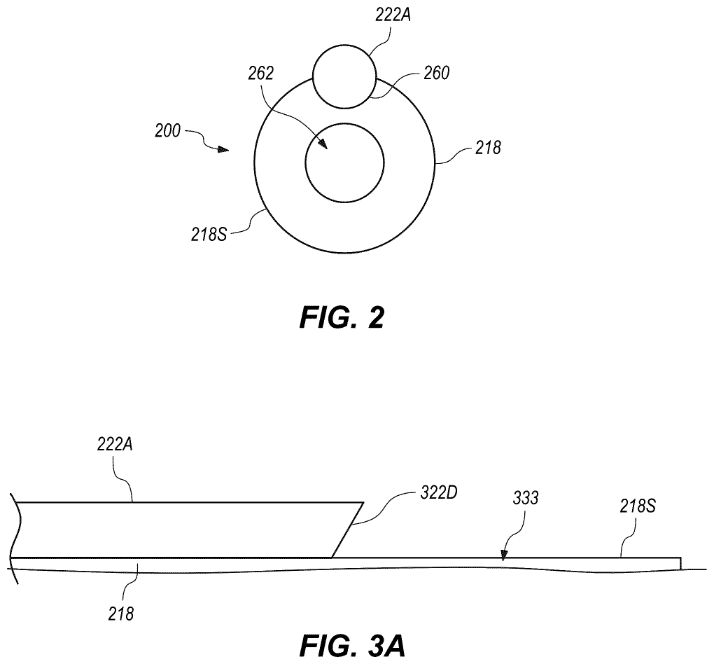

In certain embodiments, the guidewire lumen 118 can be substantially annular-shaped and/or cylindrical-shaped and can have a grooved outer surface 218S (illustrated in FIG. 2), with one or more grooves 260 (illustrated in FIG. 2) extending in a generally longitudinal direction along the guidewire lumen 118. In such embodiments, each of the energy guides 122A can be positioned, received and retained within an individual groove 260 formed along and/or into the outer surface 218S of the guidewire lumen 118. Alternatively, the guidewire lumen 118 can be formed without a grooved outer surface 218S, and the position of the energy guides 122A relative to the guidewire lumen 118 can be maintained in another suitable manner.

The catheter system 100 and/or the energy guide bundle 122 can include any number of energy guides 122A in optical communication with the energy source 124 at the proximal portion 114, and with the catheter fluid 132 within the balloon interior 146 of the balloon 104 at the distal portion 116. For example, in some embodiments, the catheter system 100 and/or the energy guide bundle 122 can include from one energy guide 122A to greater than 30 energy guides 122A. The guide distal end 122D of each of the energy guides 122A can be at any suitable or desired longitudinal position within the balloon interior 146 relative to the length 142 of the balloon 104. Alternatively, in other embodiments, the catheter system 100 and/or the energy guide bundle 122 can include greater than 30 energy guides 122A.

The energy guides 122A can have any suitable design that is useful and appropriate for purposes of enabling the generation of plasma, acoustic waves and/or pressure waves in the catheter fluid 132 within the balloon interior 146. Thus, the general description of the energy guides 122A as light guides is not intended to be limiting in any manner, except for as set forth in the claims appended hereto. More particularly, although the catheter systems 100 are often described with the energy source 124 as a light source and the one or more energy guides 122A as light guides, the catheter system 100 can alternatively include any suitable energy source 124 and energy guides 122A for purposes of generating the desired plasma in the catheter fluid 132 within the balloon interior 146. For example, in one non-exclusive alternative embodiment, the energy source 124 can be configured to provide high voltage pulses, and each energy guide 122A can include an electrode pair including spaced apart electrodes that extend into the balloon interior 146. In such embodiment, each pulse of high voltage is applied to the electrodes and forms an electrical arc across the electrodes, which, in turn, generates the plasma and forms the acoustic waves and/or pressure waves in the catheter fluid 132 that are utilized to provide the fracture force onto the vascular lesions 106A at the treatment site 106. Still alternatively, the energy source 124 and/or the energy guides 122A can have another suitable design and/or configuration, be it electrical, acoustic, pneumatic, other mechanical, etc.

As illustrated, the catheter system 100 can include one or more emitters 135 that are configured to generate plasma, acoustic waves and/or pressure waves in the catheter fluid 132 within the balloon interior 146. Each of the emitters 135 includes a guide distal end 122D of one of the energy guides 122A, which is positioned within the balloon interior 146, and a corresponding plasma generator 133 that is positioned near, but typically spaced apart from, the guide distal end 122D.

Energy from the energy source 124 is directed toward and received by the energy guide 122A, is guided through the energy guide 122A, and is then emitted from the guide distal end 122D of the energy guide 122A. The energy emitted from the guide distal end 122D is directed toward and contacts and energizes the corresponding plasma generator 133 for purposes of generating the plasma in the catheter fluid 132 within the balloon interior 146. As described in greater detail herein below, in many embodiments, the plasma generator 133 can be incorporated into the structure of and/or form a portion of the guidewire lumen 118. Stated in another manner, a portion of the guidewire lumen 118 can encompass the plasma generator 133 that is positioned near the guide distal end 122D of the energy guide 122A.

In some embodiments, the plasma generator 133 and/or another portion of the emitter 135 can be formed, at least in part, from a polymeric material. For example, in certain embodiments, the plasma generator 133 and/or the guidewire lumen 118 can be made from polymeric materials such as polyether block amide (such as PEBAX™), nylon, plastic, polyimide, or other thermoplastic, and can be designed to have a thin wall to minimize crossing profile. Polymeric fillers such as titanium dioxide, bismuth, barium sulfate, gold nanoparticles, silver nanoparticles, tungsten particles or any other metallic materials may be added to the thermoplastic to increase laser absorption and therefore improve conversion efficiency between the laser energy and acoustic output.

During use of the catheter system 100, energy (such as light energy or laser energy) that is directed out from the guide distal end 122D of the energy guide 122A comes into contact with features on the guidewire lumen 118 and/or the plasma generator 133 at a distance away from the guide distal end 122D of the energy guide 122A. The interaction of such energy with the polymeric material of the plasma generator 133 and/or the guidewire lumen 118 initiates a plasma plume which grows in size in proportion with the energy that is delivered and/or emitted from the guide distal end 122D of the energy guide 122A. The creation of the plasma plume creates a high frequency mechanical acoustic wave that is directed away from the target site of the plasma generator 133. A cavitation bubble can also be created by the acoustic wave as it propagates through the catheter fluid 132.

In certain embodiments, the energy guides 122A can include an optical fiber or flexible light pipe. The energy guides 122A can be thin and flexible and can allow light signals to be sent with very little loss of strength. The energy guides 122A can include a core surrounded by a cladding about its circumference. In some embodiments, the core can be a cylindrical core or a partially cylindrical core. The core and cladding of the energy guides 122A can be formed from one or more materials, including but not limited to one or more types of glass, silica, or one or more polymers. The energy guides 122A may also include a protective coating, such as a polymer. It is appreciated that the index of refraction of the core will be greater than the index of refraction of the cladding.

Each energy guide 122A can guide energy along its length from a guide proximal end 122P toward the guide distal end 122D, with the guide distal end 122D having at least one optical window (not shown) that is positioned within the balloon interior 146.

The energy guides 122A can assume many configurations about and/or relative to the catheter shaft 110 of the catheter 102. In some embodiments, the energy guides 122A can run parallel to the longitudinal axis 144 of the catheter shaft 110. In some embodiments, the energy guides 122A can be physically coupled to the catheter shaft 110. In other embodiments, the energy guides 122A can be disposed along a length of an outer diameter of the catheter shaft 110. In yet other embodiments, the energy guides 122A can be disposed within one or more energy guide lumens within the catheter shaft 110.

The energy guides 122A can also be disposed at any suitable positions about the circumference of the guidewire lumen 118 and/or the catheter shaft 110, and the guide distal end 122D of each of the energy guides 122A can be disposed at any suitable longitudinal position relative to the length 142 of the balloon 104 and/or relative to the length of the guidewire lumen 118 to more effectively and more precisely impart acoustic waves and/or pressure waves for purposes of disrupting the vascular lesions 106A at the treatment site 106.

In certain embodiments, the energy guides 122A can include one or more photoacoustic transducers 153, where each photoacoustic transducer 153 can be in optical communication with the energy guide 122A within which it is disposed. In some embodiments, the photoacoustic transducers 153 can be in optical communication with the guide distal end 122D of the energy guide 122A. In such embodiments, the photoacoustic transducers 153 can have a shape that corresponds with and/or conforms to the guide distal end 122D of the energy guide 122A.

The photoacoustic transducer 153 is configured to convert light energy into an acoustic wave at or near the guide distal end 122D of the energy guide 122A. The direction of the acoustic wave can be tailored by changing an angle of the guide distal end 122D of the energy guide 122A.

In certain embodiments, the photoacoustic transducers 153 disposed at the guide distal end 122D of the energy guide 122A can assume the same shape as the guide distal end 122D of the energy guide 122A. For example, in certain non-exclusive embodiments, the photoacoustic transducer 153 and/or the guide distal end 122D can have a conical shape, a convex shape, a concave shape, a bulbous shape, a square shape, a stepped shape, a half-circle shape, an ovoid shape, and the like. The energy guide 122A can further include additional photoacoustic transducers 153 disposed along one or more side surfaces of the length of the energy guide 122A.

In some embodiments, the energy guides 122A can further include one or more diverting structures or “diverters” (not shown in FIG. 1), such as within the energy guide 122A and/or near the guide distal end 122D of the energy guide 122A, that are configured to direct energy from the energy guide 122A at an angle away from the guide distal end 122D of the energy guide 122A. For example, in certain embodiments, the diverting structure can direct energy from the energy guide 122A toward a side surface which can be located at or near the guide distal end 122D of the energy guide 122A, before the energy is directed toward the balloon wall 130. Additionally, or in the alternative, the diverting structure can direct energy from the energy guide 122A at an angle toward the outer surface 218S of the guidewire lumen 118, which, as noted, can encompass the plasma generator 133 and can be formed from one or more polymeric materials in certain embodiments.

A diverting structure can include any structure of the system that diverts energy from the energy guide 122A away from its axial path, such as toward a side surface of the energy guide 122A. The energy guides 122A can each include one or more optical windows disposed along the longitudinal or circumferential surfaces of each energy guide 122A and in optical communication with a diverting structure. Stated in another manner, the diverting structures can have any suitable structural configuration that is configured to direct energy in the energy guide 122A away from its axial path and/or toward a side surface that is at or near the guide distal end 122D, where the side surface is in optical communication with an optical window. The optical windows can include a portion of the energy guide 122A that allows energy to exit the energy guide 122A from within the energy guide 122A, such as a portion of the energy guide 122A lacking a cladding material on or about the energy guide 122A.

Examples of the diverting structures suitable for use include a reflecting element, a refracting element, and a fiber diffuser. The diverting structures suitable for focusing energy away from the tip of the energy guides 122A can include, but are not to be limited to, those having a convex surface, a gradient-index (GRIN) lens, and a mirror focus lens. Upon contact with the diverting structure, the first energy can be diverted within the energy guide 122A to one or more of the plasma generator 133 that is positioned near, but typically spaced apart from, the guide distal end 122D of the energy guide 122A, and the photoacoustic transducer 154 that is in optical communication with a side surface of the energy guide 122A. When utilized, the plasma generator 133 receives energy emitted from the guide distal end 122D of the energy guide 122A to generate plasma in the catheter fluid 132 within the balloon interior 146, which, in turn, causes the creation of plasma bubbles, acoustic waves and/or pressure waves that can be directed away from the side surface of the energy guide 122A and toward the balloon wall 130. Additionally, or in the alternative, when utilized, the photoacoustic transducer 154 converts light energy into an acoustic wave that extends away from the side surface of the energy guide 122A.

The source manifold 136 can be positioned at or near the proximal portion 114 of the catheter system 100. The source manifold 136 can include one or more proximal end openings that can receive the plurality of energy guides 122A of the energy guide bundle 122, the guidewire 112, and/or an inflation conduit 140 that is coupled in fluid communication with the fluid pump 138. The catheter system 100 can also include the fluid pump 138 that is configured to inflate the balloon 104 with the catheter fluid 132 as needed.

As noted above, in the embodiment illustrated in FIG. 1, the system console 123 includes one or more of the energy source 124, the power source 125, the system controller 126, the GUI 127, and the multiplexer 128. Alternatively, the system console 123 can include more components or fewer components than those specifically illustrated in FIG. 1. For example, in certain non-exclusive alternative embodiments, the system console 123 can be designed without the GUI 127 and/or the multiplexer 128. Still alternatively, one or more of the energy source 124, the power source 125, the system controller 126, the GUI 127 and the multiplexer 128 can be provided within the catheter system 100 without the specific need for the system console 123.

As shown, the system console 123, and the components included therewith, is operatively coupled to the catheter 102, the energy guide bundle 122, and the remainder of the catheter system 100. For example, in some embodiments, as illustrated in FIG. 1, the system console 123 can include a console connection aperture 148 (also sometimes referred to generally as a “socket” or a “console receptacle”) by which the energy guide bundle 122 is mechanically coupled to the system console 123. In such embodiments, the energy guide bundle 122 can include an optical connector assembly having a guide coupling housing 150 (which can generally include one or more ferrules, and which is also sometimes referred to generally as a “connector housing”) that houses a portion, such as the guide proximal end 122P, of each of the energy guides 122A. At least a portion of the guide coupling housing 150 is configured to fit and be selectively retained within the console connection aperture 148 to provide the mechanical coupling between the energy guide bundle 122 and the system console 123, as well as helping to provide an optical coupling between the energy source 124 and the energy guides 122A of the energy guide bundle 122.

The energy guide bundle 122 can also include a guide bundler 152 (or “shell”) that brings each of the individual energy guides 122A closer together so that the energy guides 122A and/or the energy guide bundle 122 can be in a more compact form as it extends with the catheter 102 into the blood vessel 108 during use of the catheter system 100.

The energy source 124 can be selectively and/or alternatively coupled in optical communication with each of the energy guides 122A, such as to the guide proximal end 122P of each of the energy guides 122A, in the energy guide bundle 122. In particular, the energy source 124 is configured to generate energy in the form of a source beam 124A, such as a pulsed source beam, that can be selectively and/or alternatively directed to and received by each of the energy guides 122A in the energy guide bundle 122. More specifically, as illustrated, the source beam 124A from the energy source 124 is directed through the multiplexer 128 such that individual guide beams 124B (or “multiplexed beams”) can be selectively and/or alternatively directed into and received by each of the energy guides 122A in the energy guide bundle 122. In particular, each pulse of the energy source 124 and/or each pulse of the source beam 124A can be directed through the multiplexer 128 to generate a separate guide beam 124B that is selectively and/or alternatively directed onto one of the energy guides 122A in the energy guide bundle 122. As such, the energy source 124, through use and/or application of the multiplexer 128, can be utilized to energize any of the emitters 135 that may be included within the catheter system 100. Alternatively, the catheter system 100 can include more than one energy source 124. For example, in one non-exclusive alternative embodiment, the catheter system 100 can include a separate energy source 124 for each of the energy guides 122A in the energy guide bundle 122.

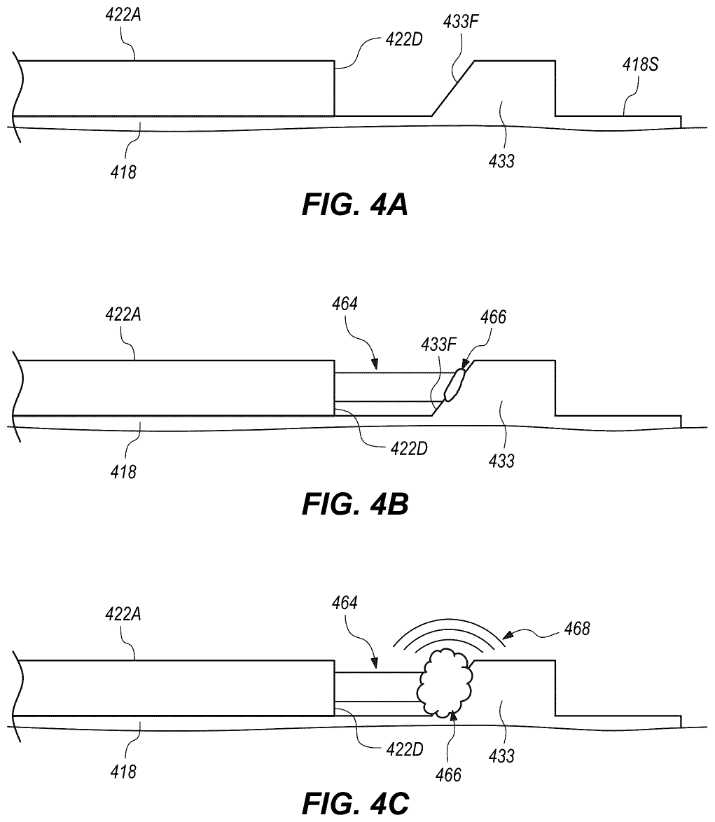

The energy source 124 can have any suitable design. In certain embodiments, the energy source 124 can be configured to provide sub-millisecond pulses of energy from the energy source 124 that are focused onto a small spot in order to couple it into the guide proximal end 122P of the energy guide 122A. Such pulses of energy are then directed and/or guided along the energy guides 122A to a location within the balloon interior 146 of the balloon 104, thereby inducing plasma formation in the catheter fluid 132 within the balloon interior 146 of the balloon 104, such as via the plasma generator 133 that can include and/or incorporate any suitable structure that is located at or near the guide distal end 122D of the energy guide 122A. In many embodiments, the plasma generator 133 can be positioned slightly spaced apart from the guide distal end 122D of the energy guide 122A. In certain embodiments, the plasma generator 133 can be provided as a portion of the outer surface 218S of the guidewire lumen 118 that is formed from one or more polymeric materials, such as within the grooves 260 that can be formed into the outer surface 218S of the guidewire lumen 118. Alternatively, in other embodiments, the plasma generator 133 can be provided in the form of a backstop-type structure with an angled face 433F (illustrated in FIG. 4A) that can be integrally formed as part of the guidewire lumen 118 and can be positioned within the grooves 260 that can be formed into the outer surface 218S of the guidewire lumen 118. As noted, the guidewire lumen 118 and/or the plasma generator 133 can be formed from one or more polymeric materials and the backstop-type plasma generator 133 can redirect energy emitted from the guide distal end 122D toward the balloon wall 130 of the balloon 104 and/or toward the vessel wall 108A of the blood vessel 108 at the treatment site 106.

In particular, in many embodiments, the energy emitted at the guide distal end 122D of the energy guide 122A is directed toward and contacts and energizes material of the plasma generator 133, such as material of the outer surface 218S of the guidewire lumen 118 and/or material on the angled face 433F of the plasma generator 133, for purposes of generating plasma in the catheter fluid 132 within the balloon interior 146. The plasma generation ionizes and superheats the surrounding catheter fluid 132 and thus causes rapid inertial bubble formation, and imparts acoustic waves and/or pressure waves upon the treatment site 106. An exemplary plasma-induced bubble 134 is illustrated in FIG. 1.

As utilized herein, the guide distal end 122D of the energy guide 122A and a corresponding plasma generator 133 can be referred to collectively as an emitter 135. In some applications, one or more emitters 135 that are positioned at approximately the same longitudinal position within the balloon interior 146 relative to the length 142 of the balloon 104 can be referred to as an “emitter station”, such as the one or more emitter stations 180 included as part of the emitter system 131 illustrated in FIG. 1.

In various non-exclusive alternative embodiments, the sub-millisecond pulses of energy from the energy source 124 can be delivered to the treatment site 106 at a frequency of between approximately one hertz (Hz) and 5000 Hz, between approximately 30 Hz and 1000 Hz, between approximately ten Hz and 100 Hz, or between approximately one Hz and 30 Hz. Alternatively, the sub-millisecond pulses of energy can be delivered to the treatment site 106 at a frequency that can be greater than 5000 Hz or less than one Hz, or any other suitable range of frequencies.

It is appreciated that although the energy source 124 is typically utilized to provide pulses of energy, the energy source 124 can still be described as providing a single source beam 124A, such as a single pulsed source beam.

The energy source 124 suitable for use can include various types of light sources including lasers and lamps. For example, in certain non-exclusive embodiments, the energy source 124 can be an infrared laser that emits energy in the form of pulses of infrared light. Alternatively, as noted above, the energy source 124 can include any suitable type of energy source.

Suitable lasers can include short pulse lasers on the sub-millisecond timescale. In some embodiments, the energy source 124 can include lasers on the nanosecond (ns) timescale. The lasers can also include short pulse lasers on the picosecond (ps), femtosecond (fs), and microsecond (μs) timescales. It is appreciated that there are many combinations of laser wavelengths, pulse widths and energy levels that can be employed to achieve plasma in the catheter fluid 132 of the catheter 102. In various non-exclusive alternative embodiments, the pulse widths can include those falling within a range including from at least ten ns to 3000 ns, at least 20 ns to 100 ns, or at least one ns to 500 ns. Alternatively, any other suitable pulse width range can be used.

Exemplary nanosecond lasers can include those within the UV to IR spectrum, spanning wavelengths of about ten nanometers (nm) to one millimeter (mm). In some embodiments, the energy sources 124 suitable for use in the catheter systems 100 can include those capable of producing light at wavelengths of from at least 750 nm to 2000 nm. In other embodiments, the energy sources 124 can include those capable of producing light at wavelengths of from at least 700 nm to 3000 nm. In still other embodiments, the energy sources 124 can include those capable of producing light at wavelengths of from at least 100 nm to ten micrometers (μm). Nanosecond lasers can include those having repetition rates of up to 200 KHz.

In some embodiments, the laser can include a Q-switched thulium:yttrium-aluminum-garnet (Tm:YAG) laser. In other embodiments, the laser can include a neodymium:yttrium-aluminum-garnet (Nd:YAG) laser, holmium:yttrium-aluminum-garnet (Ho:YAG) laser, erbium:yttrium-aluminum-garnet (Er:YAG) laser, excimer laser, helium-neon laser, carbon dioxide laser, as well as doped, pulsed, fiber lasers.

In still other embodiments, the energy source 124 can include a plurality of lasers that are grouped together in series. In yet other embodiments, the energy source 124 can include one or more low energy lasers that are fed into a high energy amplifier, such as a master oscillator power amplifier (MOPA). In still yet other embodiments, the energy source 124 can include a plurality of lasers that can be combined in parallel or in series to provide the energy needed to create the plasma bubble 134 in the catheter fluid 132.

The catheter system 100 can generate acoustic waves and/or pressure waves having maximum pressures in the range of at least one megapascal (MPa) to 100 MPa. The maximum pressure generated by a particular catheter system 100 will depend on the energy source 124, the absorbing material, the bubble expansion, the propagation medium, the balloon material, and other factors. In various non-exclusive alternative embodiments, the catheter systems 100 can generate acoustic waves and/or pressure waves having maximum pressures in the range of at least approximately two MPa to 50 MPa, at least approximately two MPa to 30 MPa, or approximately at least 15 MPa to 25 MPa.

The acoustic waves and/or pressure waves can be imparted upon the treatment site 106 from a distance within a range from at least approximately 0.1 millimeters (mm) to greater than approximately 25 mm extending radially from the energy guides 122A when the catheter 102 is placed at the treatment site 106. In various non-exclusive alternative embodiments, the acoustic waves and/or pressure waves can be imparted upon the treatment site 106 from a distance within a range from at least approximately ten mm to 20 mm, at least approximately one mm to ten mm, at least approximately 1.5 mm to four mm, or at least approximately 0.1 mm to ten mm extending radially from the energy guides 122A when the catheter 102 is placed at the treatment site 106. In other embodiments, the acoustic waves and/or pressure waves can be imparted upon the treatment site 106 from another suitable distance that is different than the foregoing ranges. In some embodiments, the acoustic waves and/or pressure waves can be imparted upon the treatment site 106 within a range of at least approximately two MPa to 30 MPa at a distance from at least approximately 0.1 mm to ten mm. In some embodiments, the acoustic waves and/or pressure waves can be imparted upon the treatment site 106 from a range of at least approximately two MPa to 25 MPa at a distance from at least approximately 0.1 mm to ten mm. Still alternatively, other suitable pressure ranges and distances can be used.

The power source 125 is electrically coupled to and is configured to provide necessary power to each of the energy source 124, the system controller 126, the GUI 127, the multiplexer 128, and the handle assembly 129. The power source 125 can have any suitable design for such purposes.

The system controller 126 is electrically coupled to and receives power from the power source 125. The system controller 126 is coupled to and is configured to control operation of each of the energy source 124, the GUI 127 and the multiplexer 128. The system controller 126 can include one or more processors or circuits for purposes of controlling the operation of at least the energy source 124, the GUI 127 and the multiplexer 128. For example, the system controller 126 can control the energy source 124 for generating pulses of energy as desired and/or at any desired firing rate. Subsequently, the system controller 126 can then control the multiplexer 128 so that the energy from the energy source 124, as the source beam 124A, can be selectively and/or alternatively directed to each of the energy guides 122A, such as in the form of individual guide beams 124B, in a desired manner.

The system controller 126 can further be configured to control operation of other components of the catheter system 100, such as the positioning of the catheter 102, the guide distal end 122D of the energy guides 122A, and/or the emitters 135 adjacent to the treatment site 106, the inflation of the balloon 104 with the catheter fluid 132, etc. Further, or in the alternative, the catheter system 100 can include one or more additional controllers that can be positioned in any suitable manner for purposes of controlling the various operations of the catheter system 100. For example, in certain embodiments, an additional controller and/or a portion of the system controller 126 can be positioned and/or incorporated within the handle assembly 129.

The GUI 127 is accessible by the user or operator of the catheter system 100. The GUI 127 is electrically connected to the system controller 126. With such design, the GUI 127 can be used by the user or operator to ensure that the catheter system 100 is effectively utilized to impart pressure onto and induce fractures into the vascular lesions 106A at the treatment site 106. The GUI 127 can provide the user or operator with information that can be used before, during and after use of the catheter system 100. In one embodiment, the GUI 127 can provide static visual data and/or information to the user or operator. In addition, or in the alternative, the GUI 127 can provide dynamic visual data and/or information to the user or operator, such as video data or any other data that changes over time during use of the catheter system 100. In various embodiments, the GUI 127 can include one or more colors, different sizes, varying brightness, etc., that may act as alerts to the user or operator. Additionally, or in the alternative, the GUI 127 can provide audio data or information to the user or operator. The specifics of the GUI 127 can vary depending upon the design requirements of the catheter system 100, or the specific needs, specifications and/or desires of the user or operator.

The multiplexer 128 is configured to selectively and/or alternatively direct energy from the energy source 124 to each of the energy guides 122A in the energy guide bundle 122. More particularly, the multiplexer 128 is configured to receive energy from the energy source 124, such as in the form of a single source beam 124A from a single laser source, and selectively and/or alternatively direct such energy in the form of individual guide beams 124B, as desired, to each of the energy guides 122A in the energy guide bundle 122. As such, the multiplexer 128 enables a single energy source 124 to be channeled separately in any desired sequence or pattern through a plurality of energy guides 122A such that the catheter system 100 is able to impart pressure onto and induce fractures in vascular lesions 106A at the treatment site 106 within or adjacent to a vessel wall 108A of the blood vessel 108 in a desired manner. As shown, in certain embodiments, the catheter system 100 can include one or more optical elements 147 for purposes of directing the energy, such as the source beam 124A, from the energy source 124 to the multiplexer 128.

The multiplexer 128 can have any suitable design for purposes of selectively and/or alternatively directing the energy from the energy source 124 to each of the energy guides 122A of the energy guide bundle 122.

As shown in FIG. 1, the handle assembly 129 can be positioned at or near the proximal portion 114 of the catheter system 100, and/or near the source manifold 136. In this embodiment, the handle assembly 129 is coupled to the balloon 104 and is positioned spaced apart from the balloon 104. Alternatively, the handle assembly 129 can be positioned at another suitable location.

The handle assembly 129 is attached to the catheter shaft 110 and is handled and used by the user or operator to operate, position and control the catheter 102. The design and specific features of the handle assembly 129 can vary to suit the design requirements of the catheter system 100. In the embodiment illustrated in FIG. 1, the handle assembly 129 is separate from, but in electrical and/or fluid communication with one or more of the system controller 126, the energy source 124, the fluid pump 138, and the GUI 127.

In some embodiments, the handle assembly 129 can integrate and/or include at least a portion of the system controller 126 within an interior of the handle assembly 129. For example, as shown, in certain such embodiments, the handle assembly 129 can include circuitry 155, which is electrically coupled between catheter electronics and the system console 123, and which can form at least a portion of the system controller 126. In one embodiment, the circuitry 155 can include a printed circuit board having one or more integrated circuits, or any other suitable circuitry. In an alternative embodiment, the circuitry 155 can be omitted, or can be included within the system controller 126, which in various embodiments can be positioned outside of the handle assembly 129, such as within the system console 123. It is understood that the handle assembly 129 can include fewer or additional components than those specifically illustrated and described herein.

The emitter system 131 includes one or more emitter stations 180, with each emitter station 180 including one or more emitters 135. As noted, each of the emitters 135 includes a guide distal end 122D of one of the energy guides 122A, and a corresponding plasma generator 133. As referred to herein, the “plasma generator” can include and/or incorporate any suitable type of structure that is located at or near the guide distal end 122D of the energy guide 122A. In some embodiments, the plasma generator 133 can be encompassed within and/or incorporated into a portion of the guidewire lumen 118. For example, the plasma generator 133 can be encompassed and/or incorporated within a portion of the outer surface 218S of the guidewire lumen 118 and/or within the materials, such as polymeric materials in many embodiments, which are utilized to form at least a portion of the outer surface 218S of the guidewire lumen 118. Alternatively, in certain embodiments, the plasma generator 133 can be provided in the form of a backstop-type structure with an angled face 433F that can be encompassed within and/or incorporated into the structure of the guidewire lumen 118 and that redirects energy emitted from the guide distal end 122D toward the balloon wall 130 of the balloon 104 and/or toward the vessel wall 108A of the blood vessel 108 at the treatment site 106.

Each of the emitters 135 is configured to selectively receive energy from the energy source 124, under control of the system controller 126 and as directed by the multiplexer 128, and emit the energy from the guide distal end 122D toward the plasma generator 133. The energy emitted from the guide distal end 122D impinges upon and energizes material of the plasma generator 133, such as material on the outer surface 218S of the guidewire lumen 118 and/or on the angled face 433F of the plasma generator 133, for purposes of generating plasma in the catheter fluid 132 within the balloon interior 146. The plasma generation ionizes and/or superheats the surrounding catheter fluid 132 and thus causes rapid inertial bubble formation, and imparts acoustic waves and/or pressure waves upon the treatment site 106.

The emitter 135 and/or the plasma generator 133 can be formed from any suitable materials. For example, in certain non-exclusive embodiments, as noted above, the emitter 135 and/or the plasma generator 133 can be formed, at least in part, from one or more polymers, polymeric materials and/or plastics, such as polyether block amide (such as PEBAX™), polyimide, nylon or other suitable thermoplastic material. In some embodiments, the emitter 135 and/or the plasma generator 133, such as the guidewire lumen 118 in certain embodiments and/or a separate structure for the plasma generator 133, is configured to have a thin wall to minimize crossing profile. The emitter 135 and/or the plasma generator 133 can also include polymeric fillers such as titanium dioxide, bismuth, barium sulfate, gold nanoparticles, silver nanoparticles, tungsten particles or any other metallic materials may also be added to the polymeric or thermoplastic materials to increase laser absorption and therefore better optimize conversion efficiency between the laser energy and acoustic output.

Alternatively, the emitter 135 and/or the plasma generator 133 can be formed from one or more metallics and/or metal alloys having relatively high melting temperatures, such as titanium, stainless steel, tungsten, tantalum, platinum, molybdenum, niobium, iridium, etc. Still alternatively, the emitter 135 and/or the plasma generator 133 can be formed from at least one of magnesium oxide, beryllium oxide, tungsten carbide, titanium nitride, titanium carbonitride, and titanium carbide. Yet alternatively, the emitter 135 and/or the plasma generator 133 can be formed from at least one of diamond CVD and diamond. In other embodiments, the emitter 135 and/or the plasma generator 133 can be formed from a transition metal, an alloy metal, or a ceramic material. Still alternatively, the emitter 135 and/or the plasma generator 133 can be formed from any other suitable materials. It is further appreciated that the emitters 135 and/or the plasma generator 133 can be formed from any suitable combination of any of the materials noted above.

The catheter system 100 can also include the fluid pump 138 that is configured to inflate the balloon 104 with the catheter fluid 132 as needed.

As with all embodiments illustrated and described herein, various structures may be omitted from the figures for clarity and ease of understanding. Further, the figures may include certain structures that can be omitted without deviating from the intent and scope of the invention.

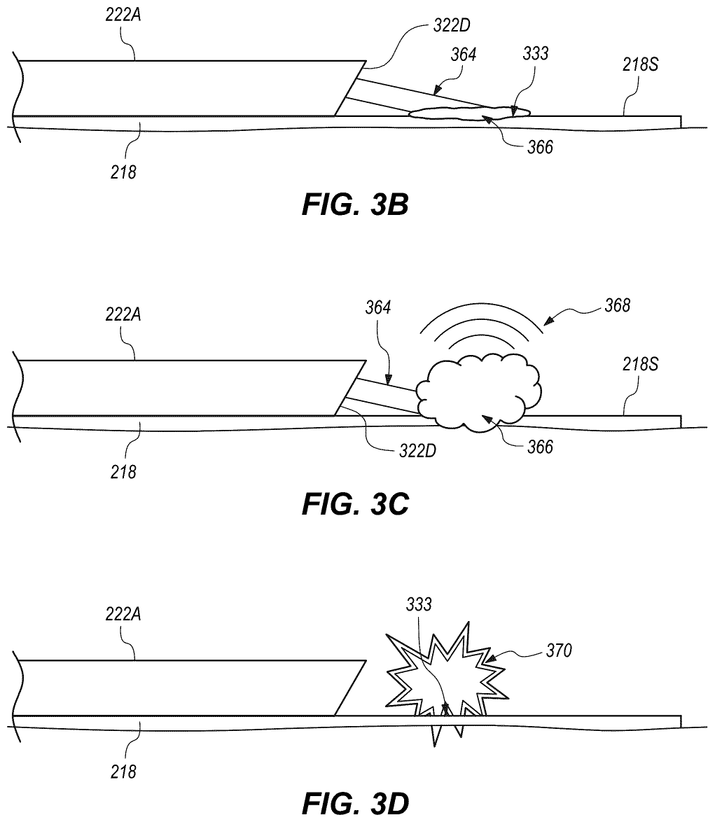

FIG. 2 is a simplified schematic end view illustration of a portion of an embodiment of the catheter system 200 including an embodiment of the guidewire lumen 218 and an energy guide 222A that is positioned adjacent to the guidewire lumen 218. More specifically, as shown in this embodiment, the guidewire lumen 218 includes a groove 260 (or channel) that is formed along and/or into an outer surface 218S of the guidewire lumen 218. The groove 260 is configured to extend in a generally longitudinal direction along at least a portion of the length of the guidewire lumen 218. As further illustrated, at least a portion of the energy guide 222A, such as at least the guide distal end 322D (illustrated in FIG. 3A) of the energy guide 222A, can be positioned, received and retained within the groove 260. With such design, the energy guide 222A can be effectively maintained in a fixed position relative to the guidewire lumen 218. It is appreciated that although only a single groove 260 is shown as being formed along and/or into the outer surface 218S of the guidewire lumen 218, the guidewire lumen 218 can be configured to include any suitable number of grooves 260 that are each configured to receive and retain a separate energy guide therein.

Alternatively, the guidewire lumen 218 can be formed without a grooved outer surface 218S, and the position of the energy guide 222A relative to the guidewire lumen 118 can be maintained in another suitable manner.

FIG. 2 further illustrates how the guidewire lumen 218 is substantially annular-shaped and/or cylindrical-shaped and defines a lumen conduit 262 through which the guidewire 112 (illustrated in FIG. 1) extends. Alternatively, the guidewire lumen 218 can have another suitable shape.

FIG. 3A is a simplified schematic side view illustration of a portion of the guidewire lumen 218 and a portion of the energy guide 222A illustrated in FIG. 2, and an embodiment of the plasma generator 333 which can be incorporated into the guidewire lumen 218. As illustrated in this embodiment, the guide distal end 322D of the energy guide 222A is angled relative to the outer surface 218S of the guidewire lumen 218, i.e. the guide distal end 322D of the energy guide 222A is not perpendicular to the outer surface 218S of the guidewire lumen 218. With such design, energy that is emitted from the guide distal end 322D of the energy guide 222A would be naturally directed at a slightly downward angle toward the outer surface 218S of the guidewire lumen 218. More specifically, the energy that is emitted from the guide distal end 322D of the energy guide 222A is directed at a slightly downward angle toward a portion of the outer surface 218S of the guidewire lumen 218 that encompasses and/or incorporates the plasma generator 333. It is appreciated that the angle of the guide distal end 322D relative to perpendicular to the outer surface 218S of the guidewire lumen 218 can be any suitable angle, such as between approximately five degrees and 45 degrees in certain non-exclusive embodiments.

In various embodiments, as noted above, the portion of the outer surface 218S of the guidewire lumen 218 that encompasses and/or incorporates the plasma generator 333, and/or the guidewire lumen 218 in its entirety, can be formed from any suitable polymeric materials. For example, in certain non-exclusive embodiments, the portion of the outer surface 218S of the guidewire lumen 218 that encompasses and/or incorporates the plasma generator 333 can be formed at least partially from one or more polymers, polymeric materials and/or plastics, such as polyether block amide (such as PEBAX™), polyimide, nylon or other suitable thermoplastic material. In some embodiments, the plasma generator 333 can also include polymeric fillers such as titanium dioxide, bismuth, barium sulfate, gold nanoparticles, silver nanoparticles, tungsten particles or any other metallic materials that may be added to the polymeric or thermoplastic materials to increase laser absorption and therefore better optimize conversion efficiency between the laser energy and acoustic output.

It is appreciated that the outer surface 218S of the guidewire lumen 218 shown in FIG. 3A as functioning as the plasma generator 333 can be part of the outer surface 218S of the guidewire lumen 218 that is found within the groove 260 (illustrated in FIG. 2) that is formed along and/or into the outer surface 218S of the guidewire lumen 218. Alternatively, the outer surface 218S of the guidewire lumen 218 functioning as the plasma generator 333 can be another part of the outer surface 218S of the guidewire lumen 218.

It is further appreciated that in other embodiments, the guide distal end 322D of the energy guide 222A can be square-cut and perpendicular to the outer surface 218S of the guidewire lumen 218, but instead can include any suitable type of diverting structure that is configured such that the energy that is emitted from the guide distal end 322D of the energy guide 222A is again directed at a slightly downward angle toward the portion of the outer surface 218S of the guidewire lumen 218 that encompasses and/or incorporates the plasma generator 333.

FIG. 3B is a simplified schematic side view illustration of the portion of the guidewire lumen 218, the plasma generator 333, and the portion of the energy guide 222A illustrated in FIG. 3A, which further illustrates plasma plume initiation. More specifically, as illustrated, energy 364, such as light energy or laser energy in certain embodiments, is emitted from the guide distal end 322D of the energy guide 222A and is directed at a slightly downward angle toward the portion of the outer surface 218S of the guidewire lumen 218 that encompasses and/or incorporates the plasma generator 333. The portion of the outer surface 218S of the guidewire lumen 218 that encompasses and/or incorporates the plasma generator 333 is shown as being spaced apart from the guide distal end 322D of the energy guide 222A.

When the energy 364 emitted from the guide distal end 322D of the energy guide 222A contacts or otherwise impinges on and energizes material of the plasma generator 333, formation of a plasma plume 366 is initiated substantially adjacent to the plasma generator 333 and/or the outer surface 218S of the guidewire lumen 218. Stated in another manner, the interaction of the energy 364 emitted from the guide distal end 322D of the energy guide 222A with the polymeric material incorporated within the plasma generator 333 initiates the plasma plume 366, which grows in size in proportion with the energy 364 that is delivered to the plasma generator 333.

FIG. 3C is a simplified schematic side view illustration of the portion of the guidewire lumen 218, the plasma generator 333 (illustrated more clearly in FIG. 3B), and the portion of the energy guide 222A illustrated in FIG. 3A, which further illustrates plasma plume generation and resulting high-frequency acoustic waves. More specifically, FIG. 3C again illustrates that energy 364, such as light energy or laser energy in certain embodiments, is emitted from the guide distal end 322D of the energy guide 222A and is directed at a slightly downward angle toward the portion of the outer surface 218S of the guidewire lumen 218 that encompasses and/or incorporates the plasma generator 333.

When the energy 364 emitted from the guide distal end 322D of the energy guide 222A contacts or otherwise impinges on and energizes the polymeric material of the plasma generator 333, formation of a plasma plume 366 is initiated substantially adjacent to the plasma generator 333 and/or the outer surface 218S of the guidewire lumen 218. The plasma plume 366 then continues to grow in size in proportion with the energy 364 that is delivered to the plasma generator 333. Subsequently, as further illustrated in FIG. 3C, the creation of the plasma plume 366 creates a high-frequency mechanical acoustic wave 368 that is directed away from the plasma generator 333 and toward the vascular lesions 106A (illustrated in FIG. 1) at the treatment site 106 (illustrated in FIG. 1). The acoustic wave 368 then imparts pressure adjacent to the vascular lesions 106A at the treatment site 106 in order to induce fractures in the vascular lesions 106A at the treatment site 106.

FIG. 3D is a simplified schematic side view illustration of the portion of the guidewire lumen 218, the plasma generator 333, and the portion of the energy guide 222A illustrated in FIG. 3A, which further illustrates cavitation bubble generation. As illustrated, a cavitation bubble 370 has been created by the acoustic wave 368 (illustrated in FIG. 3C) as the acoustic wave 368 propagates through the catheter fluid 132 (illustrated in FIG. 1) and away from the plasma generator 333 and toward the vascular lesions 106A (illustrated in FIG. 1) at the treatment site 106 (illustrated in FIG. 1).

FIG. 4A is a simplified schematic side view illustration of a portion of another embodiment of the guidewire lumen 418, a portion of another embodiment of the energy guide 422A, and another embodiment of the plasma generator 433 which can be incorporated into and/or integrally formed with the guidewire lumen 418. As illustrated, the guide distal end 422D of the energy guide 422A has been cut square such that the guide distal end 422D of the energy guide 422A is substantially perpendicular to the outer surface 418S of the guidewire lumen 418. FIG. 4A also illustrates that, in this embodiment, the plasma generator 433 is a structure that is positioned spaced apart from the guide distal end 422D of the energy guide 422A and is substantially adjacent to the remainder of the outer surface 418S of the guidewire lumen 418. More particularly, the plasma generator 433 is built up and/or extends generally outwardly away from the remainder of the outer surface 418S of the guidewire lumen 418 and can be positioned within the groove 260 (illustrated in FIG. 2) that is formed along and/or into the outer surface 418S of the guidewire lumen 418. As such, in many embodiments, the plasma generator 433 in this embodiment is again encompassed within a portion of the guidewire lumen 418. Alternatively, in other embodiments, the plasma generator 433 can be a separate structure that is positioned substantially adjacent to and/or secured to the guidewire lumen 418, such as within the groove 260 that is formed along and/or into the outer surface 418S of the guidewire lumen 418.

The design of the plasma generator 433 can be varied. As shown in FIG. 4A, in one embodiment, the plasma generator 433 can be provided in the form of a backstop-type structure having an angled face 433F that is angled relative to (i.e. is not parallel to) the square cut guide distal end 422D of the energy guide 422A. It is appreciated that the angle of the angled face 433F of the plasma generator 433 relative to the guide distal end 422D of the energy guide 422A can be any suitable angle, such as between approximately five degrees and 45 degrees in certain non-exclusive embodiments. Alternatively, the plasma generator 433 can have another suitable design and/or the angled face 433F can be angled to a different extent relative to the guide distal end 422D of the energy guide 422A.

In various embodiments, as noted above, the plasma generator 433, or at least the angled face 433F of the plasma generator 433, can be formed from any suitable polymeric materials. For example, in certain non-exclusive embodiments, the plasma generator 433 and/or the angled face 433F can be formed at least partially from one or more polymers, polymeric materials and/or plastics, such as polyether block amide (such as PEBAX™), polyimide, nylon or other suitable thermoplastic material. In some embodiments, the plasma generator 433 and/or the angled face 433F can also include polymeric fillers such as titanium dioxide, bismuth, barium sulfate, gold nanoparticles, silver nanoparticles, tungsten particles or any other metallic materials that may be added to the polymeric or thermoplastic materials to increase laser absorption and therefore better optimize conversion efficiency between the laser energy and acoustic output. It is appreciated that since, in many embodiments, the plasma generator 433 is physically incorporated into the physical structure of the guidewire lumen 418, the guidewire lumen 418 as a whole can be formed from any such polymeric materials.

FIG. 4B is a simplified schematic side view illustration of the portion of the guidewire lumen 418, the plasma generator 433, and the portion of the energy guide 422A illustrated in FIG. 4A, which further illustrates plasma plume initiation. More specifically, as illustrated, energy 464, such as light energy or laser energy in certain embodiments, is emitted from the guide distal end 422D of the energy guide 422A and is directed toward the angled face 433F of the plasma generator 433. The angled face 433F of the plasma generator 433 is again shown as being spaced apart from the guide distal end 422D of the energy guide 422A.

When the energy 464 emitted from the guide distal end 422D of the energy guide 422A contacts or otherwise impinges on and energizes material of the plasma generator 433, formation of a plasma plume 466 is initiated substantially adjacent to the angled face 433F of the plasma generator 433. Stated in another manner, the interaction of the energy 464 emitted from the guide distal end 422D of the energy guide 422A with the polymeric material incorporated within the angled face 433F of the plasma generator 433 initiates the plasma plume 466, which grows in size in proportion with the energy 464 that is delivered to the angled face 433F of the plasma generator 433.

FIG. 4C is a simplified schematic top view illustration of the portion of the guidewire lumen 418, the plasma generator 433, and the portion of the energy guide 422A illustrated in FIG. 4A, which further illustrates plasma plume generation and resulting high-frequency acoustic waves. More specifically, FIG. 4C again illustrates that energy 464, such as light energy or laser energy in certain embodiments, is emitted from the guide distal end 422D of the energy guide 422A and is directed toward the angled face 433F (illustrated more clearly in FIG. 4A) of the plasma generator 433.

When the energy 464 emitted from the guide distal end 422D of the energy guide 422A contacts and energizes the polymeric material of the angled face 433F of the plasma generator 433, formation of a plasma plume 466 is initiated substantially adjacent to the angled face 433F of the plasma generator 433. The plasma plume 466 then continues to grow in size in proportion with the energy 464 that is delivered to the plasma generator 433. Subsequently, as further illustrated in FIG. 4C, the creation of the plasma plume 466 creates a high-frequency mechanical acoustic wave 468 that is redirected away from the angled face 433F of the plasma generator 433 and toward the vascular lesions 106A (illustrated in FIG. 1) at the treatment site 106 (illustrated in FIG. 1). The acoustic wave 468 then imparts pressure adjacent to the vascular lesions 106A at the treatment site 106 in order to induce fractures in the vascular lesions 106A at the treatment site 106.

Although not specifically illustrated in FIG. 4C, as with the previous embodiment, a cavitation bubble can also be created by the acoustic wave 468 as the acoustic wave 468 propagates through the catheter fluid 132 (illustrated in FIG. 1) and away from the angled face 433F of the plasma generator 433 and toward the vascular lesions 106A (illustrated in FIG. 1) at the treatment site 106 (illustrated in FIG. 1).

In summary, in various embodiments, the catheter systems and related methods disclosed herein provide energy from an energy source that is guided by each of one or more energy guides and directed toward a corresponding plasma generator, which can be encompassed within and/or physically or structurally incorporated into the guidewire lumen, that is formed from one or more polymeric materials in order to create a plasma plume and associated high-frequency mechanical acoustic waves substantially adjacent to the plasma generator. More particularly, energy from the energy source is emitted from the guide distal end of the energy guide and is directed toward the corresponding plasma generator as part of an emitter. The interaction of the energy, light energy or laser energy in certain embodiments, with the polymeric material of the plasma generator initiates a plasma plume which grows in size in proportion with the energy that is delivered from the energy guide to the plasma generator. The creation of the plasma plume, in turn, creates a high-frequency mechanical acoustic wave that is directed away from the plasma generator and toward one or more vascular lesions located at a treatment site within or adjacent to a blood vessel wall within the body of the patient. The acoustic wave thereby imparts pressure onto and induces fractures in the vascular lesions at the treatment site.

In many embodiments, the guide distal end of the energy guide, and the corresponding plasma generator, which collectively make up the emitter, can be positioned in a catheter fluid that is retained within a balloon interior of a balloon that is positioned adjacent to the treatment site. The guide distal end of the energy guide and the corresponding plasma generator can be positioned in any suitable location relative to a length of the balloon to more effectively and precisely impart acoustic waves and/or pressure waves for purposes of disrupting the vascular lesions at the treatment site.

Thus, the catheter systems and related methods disclosed herein are configured to provide a means to generate acoustic waves and/or pressure waves that are designed to impart pressure onto and induce fractures in vascular lesions, such as calcified vascular lesions and/or fibrous vascular lesions. Importantly, in many embodiments, the emitters and/or the plasma generators (or the guidewire lumen in its entirety) can be formed to include one or more polymeric materials.

It should be noted that, as used in this specification and the appended claims, the singular forms “a,” “an,” and “the” include plural referents unless the content and/or context clearly dictates otherwise. It should also be noted that the term “or” is generally employed in its sense including “and/or” unless the content or context clearly dictates otherwise.

It should also be noted that, as used in this specification and the appended claims, the phrase “configured” describes a system, apparatus, or other structure that is constructed or configured to perform a particular task or adopt a particular configuration. The phrase “configured” can be used interchangeably with other similar phrases such as arranged and configured, constructed and arranged, constructed, manufactured and arranged, and the like.

The headings used herein are provided for consistency with suggestions under 37 CFR 1.77 or otherwise to provide organizational cues. These headings shall not be viewed to limit or characterize the invention(s) set out in any claims that may issue from this disclosure. As an example, a description of a technology in the “Background” is not an admission that technology is prior art to any invention(s) in this disclosure. Neither is the “Summary” or “Abstract” to be considered as a characterization of the invention(s) set forth in issued claims.

The embodiments described herein are not intended to be exhaustive or to limit the invention to the precise forms disclosed in the detailed description provided herein. Rather, the embodiments are chosen and described so that others skilled in the art can appreciate and understand the principles and practices. As such, aspects have been described with reference to various specific and preferred embodiments and techniques. However, it should be understood that many variations and modifications may be made while remaining within the spirit and scope herein.

It is understood that although a number of different embodiments of the catheter systems have been illustrated and described herein, one or more features of any one embodiment can be combined with one or more features of one or more of the other embodiments, provided that such combination satisfies the intent of the present invention.

While a number of exemplary aspects and embodiments of the catheter systems have been discussed above, those of skill in the art will recognize certain modifications, permutations, additions and sub-combinations thereof. It is therefore intended that the following appended claims and claims hereafter introduced are interpreted to include all such modifications, permutations, additions and sub-combinations as are within their true spirit and scope, and no limitations are intended to the details of construction or design herein shown.

What is claimed is:

1. A catheter system for treating a treatment site within or adjacent to a vessel wall of a blood vessel, the catheter system comprising:
an energy source that generates energy;
an energy guide that is configured to selectively receive the energy from the energy source, the energy guide including a guide distal end, the energy that is received by the energy guide being emitted from the guide distal end; and
a guidewire lumen having an outer surface, at least the guide distal end of the energy guide being positioned adjacent to the outer surface of the guidewire lumen, a portion of the guidewire lumen encompassing a plasma generator that is positioned near the guide distal end of the energy guide, the plasma generator being positionable near the treatment site, the plasma generator being formed from a polymeric material.

2. The catheter system of claim 1 wherein the outer surface of the guidewire lumen includes a groove, and at least the guide distal end of the energy guide is positioned within the groove formed along the outer surface of the guidewire lumen.

3. The catheter system of claim 1 wherein the plasma generator is at least partially formed from one of plastic, polyimide, nylon, and polyether block amide.

4. The catheter system of claim 1 wherein the plasma generator is positioned spaced apart from the guide distal end of the energy guide.

5. The catheter system of claim 1 wherein the energy that is received by the energy guide is emitted from the guide distal end and contacts the plasma generator so that plasma is generated adjacent to the plasma generator.

6. The catheter system of claim 5 wherein the plasma generation creates an acoustic wave that is directed away from the plasma generator.

7. The catheter system of claim 1 wherein a polymeric filler is added to the polymeric material of the plasma generator.

8. The catheter system of claim 7 wherein the polymeric filler includes one or more of titanium dioxide, bismuth, barium sulfate, gold nanoparticles, silver nanoparticles, and tungsten particles.

9. The catheter system of claim 1 further comprising a catheter shaft and a balloon that is coupled to the catheter shaft, the balloon including a balloon wall that defines a balloon interior, the balloon being configured to retain a catheter fluid within the balloon interior; and wherein the guide distal end of the energy guide and the plasma generator are positioned within the balloon interior.

10. The catheter system of claim 9 wherein the energy that is received by the energy guide is emitted from the guide distal end and contacts the plasma generator so that plasma is generated in the catheter fluid retained within the balloon interior.

11. The catheter system of claim 1 wherein the guide distal end of the energy guide is angled relative to the outer surface of the guidewire lumen so that the energy emitted from the guide distal end is emitted toward the outer surface of the guidewire lumen; and wherein the plasma generator is incorporated into the outer surface of the guidewire lumen.

12. The catheter system of claim 11 wherein the guide distal end of the energy guide is angled relative to the outer surface of the guidewire lumen at between approximately 5 degrees and 45 degrees relative to a flat, perpendicular configuration.

13. The catheter system of claim 1 wherein the plasma generator extends outwardly away from the outer surface of the guidewire lumen and includes an angled face so that the energy emitted from the guide distal end of the energy guide is emitted toward the angled face of the plasma generator and redirected toward the treatment site.

14. The catheter system of claim 13 wherein the angled face of the plasma generator is angled relative to the outer surface of the guidewire lumen at between approximately 5 degrees and 45 degrees relative to a flat, perpendicular configuration.

15. The catheter system of claim 1 further comprising a system controller including a processor that controls the energy source so that the energy from the energy source is selectively directed to the energy guide.

16. The catheter system of claim 1 wherein the energy source is a light source that generates pulses of light energy.

17. The catheter system of claim 16 wherein the light source is a laser, and the energy guide is an optical fiber.

18. A catheter system for treating a treatment site within or adjacent to a vessel wall of a blood vessel, the catheter system comprising:
an energy source that generates energy;
a catheter shaft and a balloon that is coupled to the catheter shaft, the balloon including a balloon wall that defines a balloon interior, the balloon being configured to retain a catheter fluid within the balloon interior;
an energy guide that is configured to selectively receive the energy from the energy source, the energy guide including a guide distal end that is positioned within the balloon interior, the energy that is received by the energy guide being emitted from the guide distal end; and
a guidewire lumen having an outer surface, at least the guide distal end of the energy guide being positioned adjacent to the outer surface of the guidewire lumen, a portion of the guidewire lumen encompassing a plasma generator that is positioned within the balloon interior and near the guide distal end of the energy guide, the plasma generator being positionable near the treatment site, the plasma generator being formed at least partially from a polymeric material that includes at least one of plastic, polyimide, nylon, and polyether block amide.

19. The catheter system of claim 18 wherein the energy source is a laser, and the energy guide is an optical fiber.

20. A method for treating a treatment site within or adjacent to a vessel wall of a blood vessel, the method comprising the steps of:
generating energy with an energy source;
selectively receiving the energy from the energy source with an energy guide, the energy guide including a guide distal end;
emitting the energy that is received by the energy guide from the guide distal end;
positioning at least the guide distal end of the energy guide adjacent to an outer surface of a guidewire lumen, a portion of the guidewire lumen encompassing a plasma generator that is positioned near the guide distal end of the energy guide; and
positioning the plasma generator near the treatment site, the plasma generator being formed from a polymeric material.
